(19) United States
(12) Patent Application Publication
(10) Pub. No.: US 2010/0286570 A1
(43) Pub. Date: Nov. 11, 2010

(54) WEIGHT DISTRIBUTION DETERMINATION APPARATUS

(76) Inventors: Stewart Craig, Falkirk (GB); Paul Hurrion, West Midlands (GB)

Correspondence Address: JENKINS, WILSON, TAYLOR & HUNT, P. A., 3100 Tower Blvd., Suite 1200, DURHAM, NC, 27707, US

(21) Appl. No.: 12/598,340

(22) PCT Filed: May 1, 2008
(86) PCT No.: PCT/GB08/01522

§ 371 Date: Jul. 22, 2010

(30) Foreign Application Priority Data
May 2, 2007 (GB) 0708481.7

(51) Int. Cl. A61B 5/103; A43B 13/00

(52) U.S. Cl. 600/592; 36/25 R

(57) ABSTRACT

An apparatus (10) for determining the weight distribution between front and rear portions of a foot (24) of an individual standing on a surface (26). The apparatus (10) comprises a compressible elongate body member (11) which has an outer resilient housing (12) and at least one inner chamber (14). The at least one inner chamber (14) includes inflation means (22) located therein and the body member (11) is configurable to be positioned between the arch of an individual's foot (24) and the surface (26). The inflation means (22) is inflatable such that the body member (11) provides a pivot point between the front and rear portions (28, 30) of the individual's foot (24).

The present invention relates to a weight distribution determination apparatus, particularly, but not exclusively, to a weight distribution determination apparatus for use in sports.

A number of sporting activities, such as golf, skiing, archery etc., require the individual to have an even distribution of weight, for example, between the heel and toe of each foot. However, due to the body's difficulty in perceiving it's weight distribution, an individual cannot tell if their weight is evenly distributed.

Apparatus such as pressure platforms are typically used to display an individual's weight distribution. While such apparatus can accurately determine an individual's weight distribution, they are limited in that they can only provide the information to the individual through data presentation, i.e. numerical values, diagrams, graphs etc. Therefore, an individual wishing to correct their weight distribution so that it is evenly balanced must go through a laborious and time consuming process of making incremental adjustments to their stance, or posture, and waiting for the apparatus to calculate and display the new weight distribution to them.

Furthermore, such pressure platforms are expensive and not readily accessible for individuals.

It is a object of the present invention to provide a weight distribution determination apparatus which obviates or mitigates one or more of the disadvantages referred to above.

According to a first aspect of the present invention there is provided an apparatus for determining the weight distribution between front and rear portions of a foot of an individual standing on a surface, the apparatus comprising:
a compressible elongate body member having an outer resilient housing and an inner chamber; and
inflation means located within the inner chamber, wherein, in use, the body member is configured to be positioned between the arch of an individual's foot and the surface and the inflation means is inflated such that the body member provides a pivoting point between the front and rear portions of the individual's foot

Preferably, the apparatus further comprises weight distribution measuring means for measuring the weight distribution between the front and rear portions of the foot.

Preferably, the weight distribution measuring means measures the pressure within the inflation means.

Preferably, the weight distribution measuring means includes display means for displaying the weight distribution between the front and rear portions of the foot.

Preferably, the display means includes a numerical display.

Preferably, the weight distribution measuring means includes signal generation means for generating a signal when the weight distribution between the front and rear portions of the individual's foot falls outside a predetermined range.

Preferably, the signal generation means generates an audible signal when the weight distribution between the front and rear portions of the individual's foot falls outside a predetermined range. More preferably, the signal generation means generates a visible signal when the weight distribution between the front and rear portions of the individual's foot falls outside a predetermined range.

Preferably, the inflation means comprises an inflatable bladder.

Preferably, the body member includes a flat base portion.

Preferably, the base portion is rigid.

Preferably, the base portion is made of rubber. More preferably, the base portion is made of plastic.

Preferably, the base portion comprises one or more gripping portions.

Preferably, the body member includes a protective cover.

Preferably, the protective cover is made from poly vinyl chloride.

Preferably, the body member is of sufficient length to allow it to be placed under both feet of an individual.

Preferably, the body member comprises two separate inner chambers, wherein each chamber includes inflation means located therein.

Preferably, the apparatus comprises weight distribution measuring means for measuring the weight distribution between the front and rear portions of the each foot.

Preferably, each weight distribution measuring means measures the pressure within the inflation means.

Preferably, each weight distribution measuring means includes display means for displaying the weight distribution between the front and rear portions of the foot.

Preferably, each display means includes a numerical display.

Preferably, each weight distribution measuring means includes signal generation means for generating a signal when the weight distribution between the front and rear portions of the individual's foot falls outside a predetermined range.

Preferably, the signal generation means generates an audible signal when the weight distribution between the front and rear portions of the individual's foot falls outside a predetermined range. More preferably, the signal generation means generates a visible signal when the weight distribution between the front and rear portions of the individual's foot falls outside a predetermined range.

According to a second aspect of the present invention there is provided an article of footwear including the apparatus according to the first aspect, wherein the apparatus is located between the sole of the article of footwear and the surface.

An embodiment of the present invention will now be described, by way of example only, with reference to the accompanying drawings, in which:

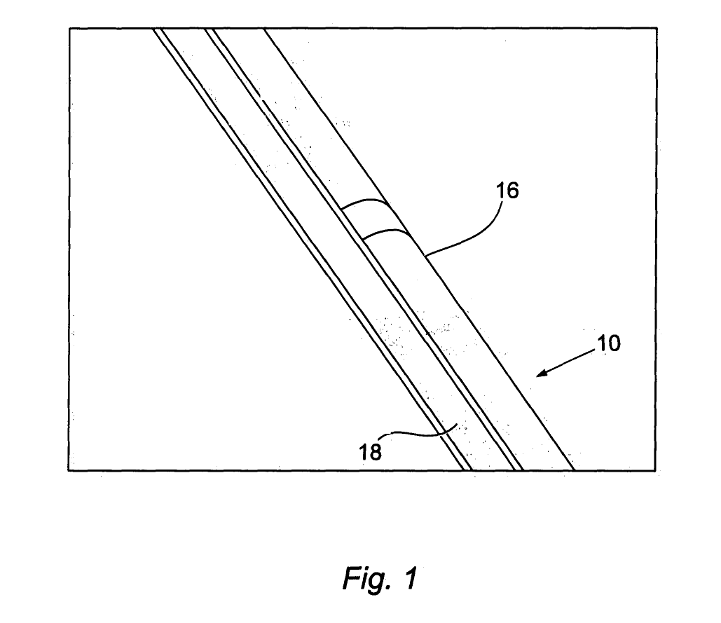

FIG. 1 is a rear perspective view of an apparatus for determining the weight distribution between front and rear portions of a foot of an individual standing on a surface;

Figure, 2 is a cross sectional end view of the apparatus of FIG. 1; and

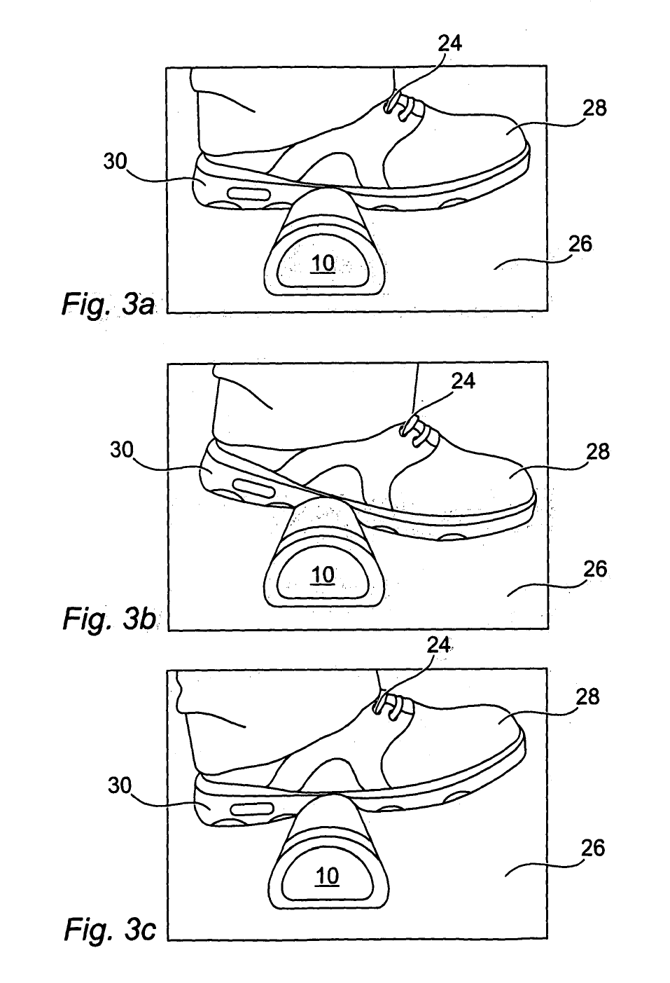

FIGS. 3a to 3c are perspective views of the apparatus of FIG. 1 in use.

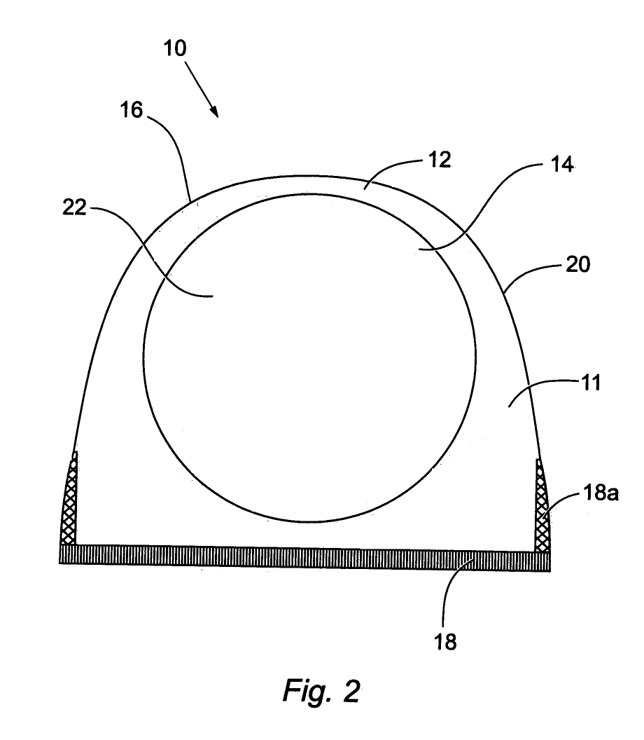

FIGS. 1 and 2 illustrate an apparatus 10 for determining the weight distribution between front and rear portions of a foot of an individual standing on a surface.

The apparatus 10 has a compressible elongate body member 11 having an outer resilient housing 12 and an inner chamber 14.

The outer resilient housing 12 defines the inner chamber 14. The housing 12 has a curved outer upper surface 16 and a substantially flat base portion 18. The base portion also has a longitudinal lip 18a which extends upwards towards the outer upper surface 16. The longitudinal lip 18a provides extra rigidity to the apparatus 10.

The outer upper surface 16 may be provided with a protective cover 20. The protective cover 20 may be made from polyvinyl chloride (PVC) such that the apparatus 10 is impervious to water etc. The housing 12 is made from a foam material. Although it should be appreciated that the housing may be made from any other suitable resilient material, e.g. rubber etc.

The base portion 18 is made from a substantially rigid material to provide rigidity to the housing 12. The base portion 18 may either be made from a rubber material, or may have an outer coating of rubber. Alternatively, the base portion 18 may be made from a plastic material. The rubber material allows the apparatus 10 to grip with a surface. The base portion 18 has one or more gripping portions (not shown) in the form of a number of grooves moulded into the rubber material.

The inner chamber 14 is also elongate and may run the length of the housing 12.

Located within the inner chamber 14 is an inflatable bladder 22 (an example of inflation means). The bladder 22 comprises a valve (not shown) which is used to inflate the bladder in a conventional manner. The bladder 22 also comprises a conventional air release valve for letting pressurised air out of the bladder 22.

With reference to FIGS. 3a to 3c, in use, the body member 11 is configured to be positioned between the arch of an individual's foot 24 and a surface 26 that the individual is standing on. Once in position, the bladder 22 is inflated to a sufficient pressure such that the body member 11 provides a pivoting point between the front and rear portion 28, 30 of an individual's foot 24.

The bladder 22 is inflated to a pressure that allows the individual to be able to determine when either the front or the rear portions 28, 30 of their foot 24 is in contact with the surface 26, or when both the front and rear portions 28, 30 are in contact with the surface 26. The apparatus 10 is therefore capable of providing kinaesthetic feedback to the individual on their weight distribution while standing.

When both the front and rear portions 28, 30 of the individual's foot 24 are in contact with the surface 26, the weight is evenly distributed therebetween and the centre of balance is directly under and through the arch of the foot 24. FIGS. 3b and 3c illustrate the conditions where the weight distribution of the individual is towards the front portion 28 of the foot 24 and towards the rear portion 30 of the foot 24, respectively. FIG. 3a illustrates the condition where the font and rear portions 28, 30 of the individual's foot 24 are in contact with the surface 26.

The apparatus 10 is of sufficient length to allow an individual to stand upon the apparatus 10 with both feet. In this case the apparatus 10 provides a pivoting point between the front and rear portions 28, 30 of both feet of the individual. The apparatus 10 therefore allows the individual to determine when their weight is evenly distributed between the front and rear portions 28, 30 of both feet.

The apparatus 10 is preferably 950 mm in length, although it should be appreciated that the apparatus 10 may be shorter or longer depending on the requirements of the individual, i.e. the type of activity the apparatus 10 is being used for.

In sports such as golf it is important to maintain a substantially even distribution of weight between the front and rear portions 28, 30 of both feet before and during swinging. An individual standing on the apparatus 10 can therefore practice their golf swing and determine their weight distribution before and during the swing. The feedback provided by the apparatus 10 allows the user to correct for uneven weight distributions, thus allowing them to achieve a more consistent swing.

The apparatus 10 further comprises a weight distribution measuring means (not shown) which measures the weight distribution between the front and rear portions 28, 30 of the foot 24. The weight distribution measuring means measures the pressure of the air in the bladder 22.

The weight distribution measuring means includes a display means for indicating the weight distribution between the front and rear portions 28, 30 of the foot 24. The display means may comprise a numerical display.

The weight distribution measuring means also includes a signal generation means for generating a signal when the weight distribution between the front and rear portions 28, 30 of the individual's foot falls outside a predetermined range, i.e. between 40% and 60%. When the weight distribution falls outside this range the signal generation means generates an audible signal to, the individual. Alternatively, the signal generation means generates a visible signal to the individual. In either case the individual is alerted that their weight distribution has fallen outside a predetermined acceptable value and that they need to correct their stance in order to maintain the ideal weight distribution.

The apparatus 10 therefore obviates or mitigates the disadvantages of previous proposals by providing kinaesthetic feedback to an individual before and during movement, for example, a golf swing. The apparatus 10 therefore avoids an individual having to going through a trial and error process in order to determine their weight distribution, as the individual gets continuous feedback from the apparatus 10. In the example of a golf swing, the individual can practice their swing using the apparatus 10 and correct for any uneven distributions of weight. Once the individual has corrected their swing they no longer need to use the apparatus 10.

Furthermore, the apparatus 10 is inexpensive and readily accessible to any individual desiring to improve their weight distribution during sporting activities.

Modifications and improvements may be made to the above without departing from the scope of the present invention. For example, although the apparatus 10 has been described above as comprising a single inner chamber 14 incorporating the inflatable bladder 22 therein, it should be appreciated that the apparatus 10 may comprise two inner chambers 14, each of which incorporates an inflatable bladder 22 therein. In this case the apparatus 10 is divided into two separate sections, each section configured to be positioned between the arch of an individual's foot. Having two separately inflatable sections allows the individual to adjust the pressure in each chamber 14 to suit a particular movement. In this case each chamber 14 comprises weight distribution measuring means for measuring the weight distribution, between the front and rear portions of the individual's foot. Also, each weight distribution measuring means measures the pressure in each bladder 22. Furthermore, each weight distribution measuring means includes display means for displaying the weight distribution between the front and rear portions 28, 30 of the foot 24. Also, the display means includes a numeric display. The weight distribution means also includes signal generation means as described above.

The apparatus 10 is designed to be able to support an individual of up to 150 kg. However, it should be appreciated that the apparatus 10 could be configured to accommodate heavier individuals.

Furthermore, although the apparatus 10 has been described above as being used in relation to golf, it should be appreciated that the apparatus 10 could be utilised in any activity, sporting or otherwise, in which an individual requires to know their weight distribution.

Also, it should be appreciated that the length of the apparatus 10 may be configured such that it may be used with one or both feet of an individual, i.e. the apparatus 10 may be short when only used with one foot, or longer such that the individual can stand upon the apparatus with their legs spread, e.g. as in a golf swing. Similarly an individual may use two separate apparatuses 10, one for each foot, if required.

Furthermore, the apparatus 10 may further comprise markers indicating where the individual should place their foot, or feet, on the apparatus 10.

Also, the height of the housing 12 may be configured to suit a particular kind of activity, sporting or otherwise. For example, when used in golf, the height of the apparatus 10 may be increased such that it can contact the cleat of the golf shoe.

Furthermore, although the apparatus 10 has been illustrated and described above as a body member which is stood upon by an individual, it should be appreciated that the apparatus 10 may be incorporated into an article of footwear, such as a golf shoe. In this case the apparatus 10 is located between the sole of the article of footwear and the surface. More specifically, the apparatus 10 is located beneath the arch of the individual's foot 24 and the surface. The apparatus 10 may be integrally formed with the article of footwear. With this arrangement, the apparatus 10 operates in the same manner as described above, i.e. providing a pivot point between the front and rear portions 28, 30 of the individual's foot 24.

Also, although the apparatus 10 has been illustrated and described above as being straight, it should be appreciated that the apparatus 10 may be configured in any shape suitable for a particular activity, e.g. curved etc.

What is claimed is:

1. An apparatus for determining the weight distribution between front and rear portions of a foot of an individual standing on a surface, the apparatus comprising:
a compressible elongate body member having an outer resilient housing and at least one inner chamber; and
inflation means located within the at least one inner chamber, wherein the body member is configurable to be positioned between the arch of an individual's foot and the surface and the inflation means is inflatable such that the body member provides a pivot point between the front and rear portions of the individual's foot.

2. An apparatus for determining weight distribution as claimed in claim 1, wherein the apparatus further comprises weight distribution measuring means for measuring the weight distribution between the front and rear portions of the foot.

3. An apparatus for determining weight distribution as claimed in claim 2, wherein the weight distribution measuring means includes display means for displaying the weight distribution between the front and rear portions of the foot.

4. An apparatus for determining weight distribution as claimed in claim 2, wherein the weight distribution measuring means includes signal generation means for generating a signal when the weight distribution between the front and rear portions of the individual's foot falls outside a predetermined range.

5. An apparatus for determining weight distribution as claimed in claim 1, wherein the inflation means comprises an inflatable bladder.

6. An apparatus for determining weight distribution as claimed in claim 1, wherein the body member comprises two separate inner chambers, wherein each chamber includes inflation means located therein.

7. An apparatus for determining weight distribution as claimed in claim 6, wherein the body member is of sufficient length to allow the apparatus to be placed under both feet of the individual.

8. An apparatus for determining weight distribution as claimed in claim 6, wherein the apparatus comprises weight distribution measuring means for measuring the weight distribution between the front and rear portions of each foot.

9. An article of footwear including the apparatus as claimed in claim 1, wherein the apparatus is located between the sole of the article of footwear and the surface.

10. An article of footwear as claimed in claim 9, wherein the apparatus is located beneath the arch of the individual's foot.

11. An article of footwear as claimed in claim 9, wherein the apparatus is integrally formed with the article of footwear.
